(19) United States
(12) Patent Application Publication
(10) Pub. No.: US 2017/0100504 A1
(43) Pub. Date: Apr. 13, 2017

(54) ACTIVATED CARBON CONTAINING WOUND DRESSING

(71) Applicant: CALGON CARBON CORPORATION, Moon Township, PA (US)

(72) Inventors: Jack Elliot TAYLOR, Cramlington (GB); Wayne LAVOCAH, South Shields (GB)

(21) Appl. No.: 15/383,130

(22) Filed: Dec. 19, 2016

Related U.S. Application Data
(63) Continuation of application No. 13/593,918, filed on Aug. 24, 2012, now abandoned.
(60) Provisional application No. 61/526,947, filed on Aug. 24, 2011.

(51) Int. Cl. A61L 15/18; A61F 13/02; A61F 13/15; A61L 15/44; A61F 13/00

(52) U.S. Cl. CPC A61L 15/18; A61L 15/44; A61F 13/00029; A61F 13/15; A61F 13/0269; A61L 2300/108; A61L 2300/404

(57) ABSTRACT

Wound dressings including activated carbon cloth or a fabric, cloth, or other flexible material containing activated carbon and methods for manufacturing and using such wound dressings to effectuate healing of wounds on humans or other animals are described herein.

### CROSS-REFERENCE TO RELATED APPLICATIONS

This application claims priority to U.S. Provisional Application No. 61/526,947, entitled “Activated Carbon Containing Wound Dressing,” filed Aug. 24, 2011, which is incorporated herein by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### SUMMARY OF THE INVENTION

Embodiments described herein include a wound dressing including a perforated impermeable first material layer, an activated carbon layer, and a perforated impermeable second material layer. In some embodiments, the activated carbon layer may be an activated carbon cloth such as a woven cloth, non-woven cloth, knitted cloth, activated carbon felt, or combinations thereof.

Other embodiments are directed to methods for making a wound dressing including the step of sealing an activated carbon layer between a perforated impermeable first material layer and the perforated impermeable second material layer.

Still other embodiments are directed to methods for treating a wound including the steps of contacting a wound dressing comprising an activated carbon layer sealed between a perforated impermeable first material layer and the perforated impermeable second material layer to a wound and applying an adsorbent material over the wound dressing. Some embodiments of methods for treating a wound include contacting a wound dressing comprising an activated carbon layer sealed between a perforated impermeable first material layer and the perforated impermeable second material layer to a wound and administering at least one therapeutic agent to the wound through the wound dressing.

### DESCRIPTION OF DRAWINGS

For a fuller understanding of the nature and advantages of the present invention, reference should be made to the following detailed description taken in connection with the accompanying drawings, in which:

FIG. 1 is a schematic illustrating an embodiment of a wound dressing including an activated carbon layer 1 between a first material layer 2 and a second material layer 3 with an adhesive layer for affixing the wound dressing to a wound or tissue surrounding a wound.

FIG. 2 is a schematic illustrating a cut-away of an embodiment of a wound dressing including an activated carbon layer 1 between a first material layer 2 and a second material layer 3 with an adhesive layer for affixing the wound dressing to a wound or tissue surrounding a wound.

FIG. 3 is a schematic showing the application of an embodiment of the wound dressing of the invention (panel 1) affixing the wound dressing to a wound (panel 2), affixing an absorbent material 5 to the wound over the wound dressing (panel 3), removing the absorbent material 5 and applying a therapeutic agent to the wound through the wound dressing (panel 4), and reaffixing a fresh absorbent material 6 over the wound dressing.

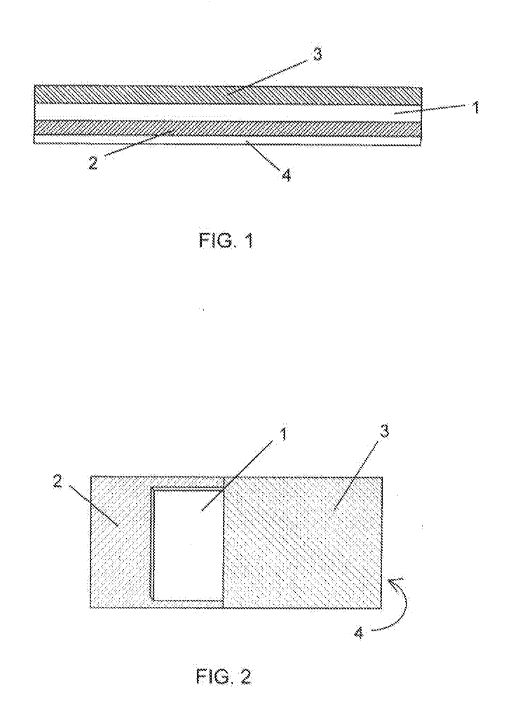

### DETAILED DESCRIPTION

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope which will be limited only by the appended claims.

It must also be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not.

“Substantially no” means that the subsequently described event may occur at most about less than 10% of the time or the subsequently described component may be at most about less than 10% of the total composition, in some embodiments, and in others, at most about less than 5%, and in still others at most about less than 1%.

Various embodiments of the invention are directed to a wound dressing including a material that includes activated carbon, methods for preparing such wound dressings, and methods for treating wounds by applying the wound dressing of various embodiments to the wound. The dressings of various embodiments can be used to treat any kind of wound such as, for example, lacerations, cuts, scrapes, abrasions, post-operative wounds, denuded skin, and burns, or other skin problems (e.g., allergies) and dressings of various sizes can be prepared such that minor wounds as well as larger wounds can be treated using the wound dressings of embodiments. In general, the dressing of such embodiments may allow transfer of air and moisture into and eat of the wound while immobilizing microbes on the activated carbon. Therefore, the activated carbon containing material in the wound dressing may provide inherent anti-microbial activity in the absence of other known anti-microbial agents, such as noble metals or pharmaceutical-type antibiotics or improved anti-microbial activity when combined with other anti-microbial agents. The wound dressings described herein can be used to treat wounds on humans or any other animal including, but not limited to, mammals, fish, reptiles, birds, and other creatures. Thus, medical and veterinary uses for the wound dressings described herein are encompassed by the invention, and such uses can be carried out by trained medical professionals, physicians, veterinarians, nurses, emergency medical technicians, and the like, or by consumers who purchase the wound dressings described herein over the counter.

In some embodiments, an activated carbon cloth or a cloth or other flexible material containing activated carbon may be applied to a wound, such as those described above, directly. Therefore, embodiments include activated carbon cloth configured and designed to be applied to a wound. As such, the activated carbon cloth may be shaped to adequately cover a wound. For example, activated carbon cloths designed and configured to be applied directly to a wound may have a square rectangular, round, butterflied, or other shape, and in other embodiments, that activated carbon cloth or other material containing activated carbon designed and configured to be applied to a wound may include sheets or tapes that can be cut or wrapped around, for example, a portion of a limb, to cover a wound on the limb. In still further embodiments, the activated carbon cloth or other material containing activated carbon designed and configured to be applied to a wound may include one or more additional components that are provided to, for example, improve healing, reduce adhesion to the wound, improve adherence to skin surrounding the wound, reduce itching, or otherwise aid in improving patient comfort. Such additional components are described below and can be incorporated into wound dressings consisting of an activated carbon cloth or a cloth or other flexible material including activated carbon.

In some embodiments, wound dressings including activated carbon cloth or a cloth or other flexible materials that contain activated carbon may include one or more additional material layers. For example, in certain embodiments, the wound dressing may include it first layer of an activated carbon cloth or a cloth or other flexible material containing activated carbon and a second layer of a flexible material covering the first layer. In such embodiments, the second layer may be gauze, an absorbent material, or another medical fabric commonly used in wound dressings, and in some embodiments, the second layer may be a flexible adhesive material that is capable of holding the first layer in position over the wound. The second layer may be provided on the first layer or the second layer may be applied separately. Therefore, some embodiments include a kit containing an activated carbon cloth or other flexible material and a flexible material configured to cover the activated carbon cloth. In operation, the activated carbon cloth or other flexible material containing material may be applied to the wound and the flexible material may be applied over the activated carbon cloth or other flexible material.

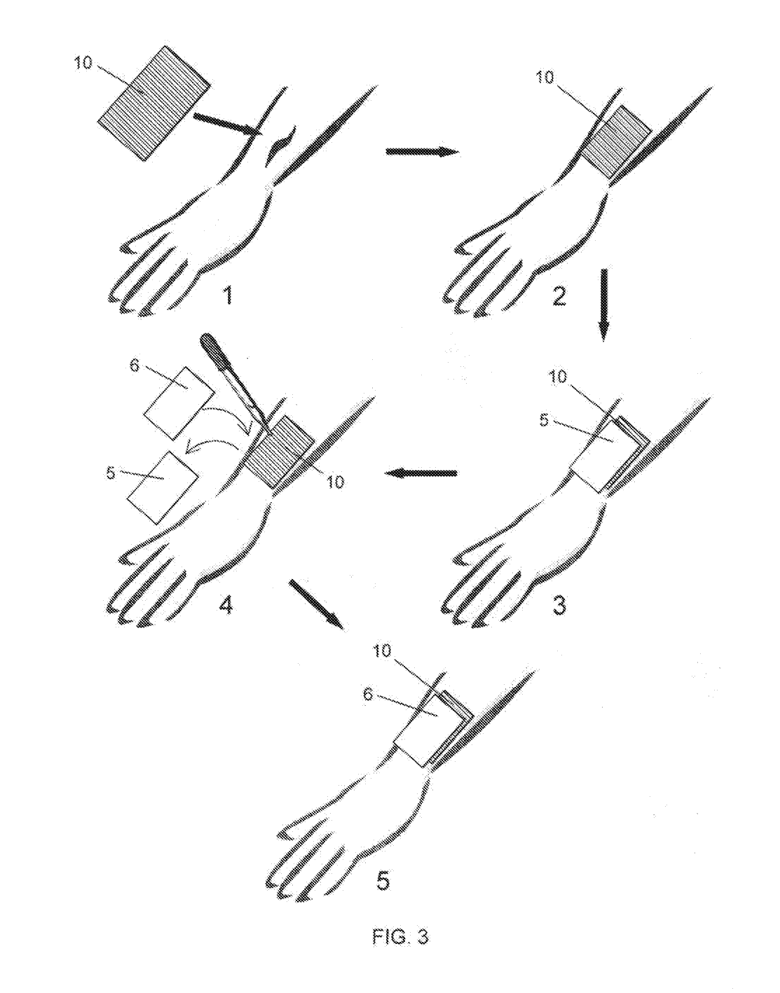

As illustrated in FIG., 1, FIG. 2, and FIG. 3, some embodiments of the invention are directed to a wound dressing including at least three material layers: a first flexible material layer 2, an activated carbon containing layer 1, and a second flexible material layer 3. The first flexible material layer 2 and the second flexible material layer 3 may be composed of any material known in the medical arts that is useful for wound dressings and may generally be composed of a material that allows air and/or fluid to pass through the wound dressing and into the wound. Non-limiting examples of such materials include gauzes and absorbent materials commonly used in wound dressings. In certain embodiments, the first flexible material layer 2 and the second flexible 3 material layer may be impermeable film, and in some embodiments, at least one of the first flexible material layer 2 and the second flexible material layer 3 may be perforated to allow the air and fluid to pass through the wound dressing and contact the wound. The wound dressings of such embodiments may be applied directly to a wound. In some embodiments, at least the first material layer 2 may include an adhesive layer that allows the wound dressing to self adhere to the wound arid/or tissue surrounding the wound, and in other embodiments, the wound dressing may be held in place over the wound using a secondary adhesive material such as, for example, tape or other adhesive wound dressings.

In some embodiments, the activated carbon containing layer 1 may be an activated carbon cloth. Embodiments are not limited by a particular type of activated carbon cloth. For example, the activated carbon cloth of various embodiments may be a woven, non-woven, knitted, or felt activated carbon cloth in various embodiments. In other embodiments, the activated carbon containing layer may include activated carbon particles, activated carbon powder, activated carbon fiber, or a combination of these materials. For example, in some embodiments, activated carbon particles may be immobilized or attached to a non-activated carbon based cloth and in other embodiments, activated carbon particles, powder, and/or fibers may be contained between sealed layers of a non-activated carbon based cloth. In still other embodiments, activated carbon particles, powder, and/or fibers may be included in a woven, non-woven, knitted, or felt activated carbon cloth material.

In some embodiments, the activated carbon containing layer may include borax, i.e., sodium borate, sodium tetraborate, or disodium tetrabotate. Without wishing to be bound by theory, the presence of borax in the activated carbon containing layer may enhance the anti-microbial activity of the activated carbon in the absence of a known anti-microbial agent. Any amount of borax may be included in the activated carbon containing layer. For example, in some embodiments, the borax may be about 0.001 wt. % to 50 wt. %, and in other embodiments, the borax may be about 0.01 wt % to 30 wt. % or about 0.1 wt. % to 25 wt. %.

As discussed above, anti-microbial activity can be achieved in the absence of other known antis-microbial agents, because as identified herein, activated carbon inherently has antimicrobial activity, and this activity can be enhanced by the addition of borax. However, in certain embodiments, the activated carbon cloth may further include one or more anti-microbial agents other than activated carbon. For example, in some embodiments, the activated carbon containing layer may include a noble metal such as silver, gold, palladium, platinum, copper, zinc, or a combination thereof, and in particular embodiments, the noble metal may be silver or zinc, in such embodiments, the noble metal may be provided at about 0.001 wt. % to about 30 wt. % or about 0.01 wt. % to about 10 wt. %.

In some embodiments, the noble metal may be provided as noble metal particles or powder, and in such embodiments, the particle size of the noble metal particles may be less than about 100 nm, less than 50 nm, and less than 25 nm. In embodiments, noble metal particles or powder are associated with the activated carbon in the activated carbon containing layer, any means known in the art can be used to create such an association including, but not limited to thermocracking, electroplating, electroless plating, or vacuum plating. In particular embodiments, noble metal particles or powder may be associated with activated carbon cloth by immersion. For example, in some embodiments, activated carbon fibers or cloth may be immersed in a solution of silver nitrate for 1 to 720 minutes at a pH of 3 to 8, which reduces the silver allowing the silver to form particles on the surface of the activated carbon fiber or cloth. Such methods may further include drying the fiber or cloth to remove residual water, which is typically carried out at a temperature of from about 25° C. to about 150° C. Silver-carrying activated carbon fiber prepared according to the above process generally results in activated carbon fibers having a BET surface area of greater than about 400 m/g, carbon content of greater than about 50 wt %, silver content of greater than about 0.001 wt %, and a density of greater than about 1.8 g/m³.

In other embodiments, a therapeutically active agent can be included in the activated carbon containing layer. The therapeutically active agent provided may be pre-adsorbed into the pore volume of the material and its subsequent release from the material may be controlled. In such embodiments, the release of the therapeutically active agent may be controlled by applying an electrical current to the material. Examples of suitable therapeutically active agents may include antibiotics, antimicrobials, sulfonamides, antiseptics, analgesics, or anesthetics and other medicaments and substances used to promote healing such as osmotic colloids, protease inhibitors, proteolytic enzymes, growth factors, steroidal or non-steroidal anti inflammatory drugs, nutrients, antioxidants, and the like, and any combination of therapeutically active agents. Examples of particular active agents that may be useful in embodiments may include, but are not limited to, acrisorcin, haloprogin, iodochlorhydroxyquin, tolnaftate, triacetin, centella asiatiea, econazole nitrate, mafenide, mupirocin, povidone iodine, chlohexidine, silver sulfadiazine, povidone iodine, silver salts, triclosan, sucralfate, quaternary ammonium salts, tetracycline, penicillins, terramycins, erythromycin, bacitracin, neomycin, polymycin B, mupirocin, clindamycin, and any mixtures thereof.

In some embodiments, the activated carbon layer may further include one or more non-toxic, pharmaceutically, and dermatologically acceptable carriers, diluents, or excipients or combinations thereof. The therapeutically active agent and/or carriers, diluents, and/or excipients may be prepared for topical use and can be in various dosage forms including, but not limited to, a gel, a paste, an ointment, a cream, an emulsion, or a suspension. For example, a suitable thickener such as, aluminum stearate or hydrogenated lanolin, or gelling agent can be added to an aqueous or oil base to formulate an ointment or cream. Examples of suitable excipients include starch, tragacanth, cellulose derivative, polyethylene glycol silicones, bentonite, silicic acid, talc, or a mixture thereof. In such embodiments, the activated carbon can be mixed with the therapeutically active agent, carrier, diluent, and/or excipient and other active components to provide the desired dosage form.

The first material layer 2 and the second material layer 3 may be composed of the same material or different materials and may be composed of any material known and used in the medical arts including synthetic and natural materials used in, for example, commercially available bandages and gauzes. In certain embodiments, the first material layer 2 and the second material layer 3 may be composed of polymeric materials. The embodiments described herein are not limited to any particular polymeric material. However, in some embodiments, the first material layer 2 and the second material layer 3 may be composed on an impermeable material, and in other embodiments, the impermeable material may be a polymer. Such polymers may generally be conformable but not substantially elastomeric, and in some embodiments, the polymer may be hydrophilic. Examples of suitable polymers include, but are not limited to, polyethylene, polypropylene, polyester, polyamides such as nylons, fluoropolymers such as polyvinylidene fluoride (PVDF) or polytetrafluoroethylene (PTFE), ethylene methyl acrylate (EMA), and mixtures thereof in other embodiments, the polymers may be drug-impermeable. Such drug impermeable materials include, for example, polyvinyl chloride, polyvinyl dichloride, polyurea, polyolefins, such but not limited to, ethylene vinylacetate copolymer, polyethylene, and polypropylene, and polyesters, such as, but not limited to, polyethylene terephtlate, and the like and combinations and mixtures of these.

The polymers of various embodiments can be provided as a single polymer layer or film or a laminate having multiple polymer layers. In general, the polymer layers can be as thin as possible consistent with the need for physical integrity during manufacture and use. Thus, in some embodiments, the first material layer 2 and the second material layer 3 may each have a basis weight of from about 1 g/m² to about 500 g/m² and in other embodiments, the first material layer 2 and the second material layer 3 may each have a basis weight of from about 10 g/m² to about 200 g/m². The thickness and basis weight of the first material layer 2 and the second material layer 3 may be the same or different. For example, in some embodiments, the first material layer 2 and the second material layer 3 may each have as basis weight that is the same, and in other embodiments, the second material layer 3 may have a basis weight that is greater than the basis weight of the first material layer 2. In such embodiments, the thicker second material layer 3 may provide abrasion resistance to the outer surface of the dressing, while the thinner first material layer 2 may provide improved flexibility for the surface of the dressing contacting the wound.

In some embodiments, the polymeric films used in the first material layer 2 and the second material layer 3 may be perforated. For example, in some embodiment, the polymer film used in the first material layer 2 and the second material layer 3 may have from about 5 perforations/cm² to about 50 perforations/cm² and in other embodiments, the polymer films may have from about 10 perforations/cm² to about 30 perforations/cm². The perforations of such embodiments may have an area of from about 0.01 mm² to about 2.0 mm² and a hole-to-land ratio, defined as the ratio of the total area of the perforations to the total area of the film less the area of the perforations both areas being viewed in plans projection, of from about 0.01 to about 1.0, about 0.05 to about 0.5 or in some embodiments, from about 0.1 to about 0.3.

Particular embodiments include a wound dressing including at least three components: (1) a double sided adhesive film which may include a release paper on at least one side to unwind the reel, (2) an activated carbon cloth such as Zonflex activated carbon cloth or a cloth or other flexible material containing activated carbon, and (3) a single sided adhesive film which may or may not have a release paper on the adhesive side. In certain embodiments, these components may be provided on reels, and manufacture of the wound dressing can be carried out by combining or contacting the leading edges of the components and laminating the components in a laminator to create the wound dressing. In some embodiments, the exposed adhesive side of double sided adhesive film may contact one side of the activated carbon cloth leaving the release paper on the other side of the double side's adhesive film as an outer exposed side of the wound dressily. The adhesive side of the single sided adhesive film may contact activated carbon cloth at the side opposite the double sided adhesive such that the non-adhesive side is exposed. Lamination may result in all width rolls of wound dressing, and in some embodiments, the laminated wound dressing may be cut into usable sized portions. The retained layer of release paper can be removed immediately before use by the user.

Additional methods for preparing the wound dressings of embodiments include preparing perforated polymer materials as discussed above. For example, in mesh perforation, a film of the polymeric material is supported on a reticulated mesh surface and heated to its softening temperature. Suction is applied through the mesh, or air is blown onto the film above the mesh resulting in an impression of the mesh onto the film and the formation of perforations in the film at the interstices of the mesh. Mesh perforation technique are described in more detail in U.S. Pat. No. 3,054,148, the entire content of which is expressly incorporated herein by reference.

In some embodiments, the first material layer 2 and the second material layer 3 may be textured. As used herein, the term “textured” indicates that the film is patterned in relief with, for example, protruding ridges or nubs. Such ridges or nubs are generally rounded and may project from about 0.1 mm to about 1.5 mm above the median plane of the film surface or in some embodiments from about 0.2 mm to about 1.0 mm above the median plane of the film. In various embodiments, either the first material layer 2 or the second material layer 3 material layer, or both the first material layer 2 and the second material layer 3, may be textured. In particular embodiments, the first material layer 2 may be textured, which may render the first material layer 2 less adherent to a wound bed.

In some exemplary embodiments, the second material layer 3 may have a smoothed perforated surface and may be designed to be the rear side (i.e., applied facing away from the wound surface) of the envelope that is smoother than the first material layer 2 designed to be the front (i.e., wound contacting) side of the envelope. For example, in some embodiments, the surface roughness or degree of texturization of the rear side of the second material layer 3 may be less than about 80% of the surface roughness or degree of texturization of the from side of the first material layer 2. In other embodiments, the degree of texturization of the rear may be less than about 70%, less than about 50%, or less than about 30%.

The textured wound contacting surface of the envelope may allow for low adherence and good wicking of liquid from the wound into the activated carbon layer 1 through the first material layer 2. In contrast, the smoother second material layer 3 on the rear side of the envelope may provide excellent adhesion to the adhesive layer on the backing sheet, while retaining perforations that enable water vapor to diffuse out through the second material layer 3 preventing saturation of the activated carbon layer 1. By the term “smoothed” is meant that the rear second material layer 3 has less surface texturing (roughness, unevenness) than the front first material layer 2 of the envelope. In some embodiments, substantially no projections on the smoothed surface will project greater than 100 μm above the median plane of the smoothed film surface, and in other embodiments, substantially no projections will project greater than 25 μm above the median plane of the smoothed film surface.

In some embodiments, the first material layer 2 and the second material layer 3 may form an envelope or island for the activated carbon layer 1. The term “envelope” or “island” as used herein means that the front and back faces of the activated carbon layer 1 are substantially covered by the first material layer 2 and the second material layer 3, such that both faces and each edge of the activated carbon layer 1 are covered by the first material layer 2 and the second material layer 3. For example, in certain embodiments as illustrated in FIG. 3, two sheets of a polymeric film may be positioned such that a first sheet is above the activated carbon layer 1 and a second sheet is below the activated carbon layer 1 covering the to and bottom faces of the activated carbon layer 1 and extending beyond the edges of the activated carbon layer 1. The two sheets can be bonded along two or more edges of each sheet to form an envelope. In other embodiments, an envelope, can be formed from a single polymer film that has been folded with the activated carbon layer 1 between the folds. The opposed longitudinal edges of the film may overlap, and the overlapping edges can be bonded together to form the envelope. Such envelopes or islands can be sealed and the polymeric film edges can be bonded using, for example, hot melt adhesives or heat bonding, and can be made by minor modification of conventional form-fill-seal equipment.

In certain embodiments, the second material layer 3 may be prepared from a material that is semi-permeable. For example, the second material layer 3 is permeable to water vapor but not permeable to liquid water, and/or the second material layer 3 may be microorganism impermeable. Suitable materials for the second material layer 3 may have a moisture vapor transmission rate (MVTR) of for example, about 300 g/m²/24 hrs. to about 5000 g/m²/24 hrs., or from about 500 g/m²/24 hrs. to about 2000 g/m.sup.2/24 hrs at about 37° C. at 100% to 10% relative humidity. The second material layer 3 of such embodiments may further have a thickness of from about 10 μm to about 1000 μm or from about 100 μm to about 500 μm.

In some embodiments, the MVTR of the dressing of various embodiments as a whole may be lower than that of second material layer 3 alone, because the polymeric film envelope partially obstructs moisture transfer through the dressing. For example, in some embodiments, the MVTR of the dressing (measured across the island portion of the dressing which includes the activated carbon layer 1) may be from about 20% to about 80% of the MVTR of the second material layer 3 alone. In other embodiments, the MVTR of the dressing may be from about 20% to about 60% or about 40% of the MVTR of the second material layer 3 alone, Without wishing to be bound by theory, such moisture vapor transmission rates may allow the wound under the dressing to heal under moist conditions without causing the skin surrounding the wound to macerate.

Various embodiments of the dressing may include an adhesive layer 4 on either the first material layer 2 or the second material layer 3 or both the first material layer 2 and the second material layer 3. In certain embodiments, an adhesive layer 4 may be provided on the first material layer to allow the first material layer to adhere to the wound and/or tissue surrounding the wound. In such embodiments, the adhesive layer 4 can be moisture vapor transmitting and/or patterned to allow passage of water vapor through the adhesive layer 4.

Any adhesive or combination of adhesives known in the medical arts may be used in the adhesive layer 4. In general, the adhesive may be moisture vapor transmitting and pressure-sensitive, meaning that it forms a bond when applied with light pressure, of the type conventionally used for island-type wound dressings. Adhesives useful in various embodiments include, but are not limited to, acrylate ester copolymers, polyvinyl ethyl ether and polyurethane pressure sensitive adhesives. The relative thickness of the adhesive layer may vary among embodiments and may be optimized based on the type of adhesive used. In exemplary embodiments, the basis weight of the adhesive layer may be from about 20 g/m² to about 250 g/m² or from about 50 g/m² to 150 g/m². In particular embodiments, the adhesive may be a polyurethane-based pressure sensitive adhesive.

In some embodiments, the adhesive layer 4 may be continuous with the first material layer 2 and/or the second material layer 3, and in other embodiments, the adhesive layer may extend outwardly from the island created by the activated carbon layer 1 and the envelope to form an adhesive-coated margin as in conventional island-type wound dressings. In some embodiments, the adhesive layer 4 may further include a release coat or a cover layer to protect the adhesive and absorbent layer before use. Various release coats and cover layers are known in the art and can be used in conjunction with embodiments of the invention. For example, in certain embodiments, the cover layer may be a silicone release-coated paper.

Generally, the wound dressings of various embodiments described above will be sterile and can be packaged in a microorganism-impermeable container such as a pouch.

Embodiments further include methods for using the wound dressings described above. The methods of various embodiments generally include the step of contacting a wound with a wound dressing 10 having a first material layer 2 and the second material layer 3 and an activated carbon layer 1 directly to a wound such that the first material layer 2 contacts and adheres to the wound, and an exemplary method is illustrated in FIG. 3. In some embodiments, the wound dressing 10 may include an adhesive layer 4 that allows the dressing to remain in place over the wound after contacting the wound, and in other embodiments, the method may include applying adhesive tape or another than of bandage over the wound dressing 10 to allow it to remain in position over the wound.

In further embodiments, the method may further include contacting the wound dressing 10 with an absorbent material 5, such as cotton or gauze, that may absorb and collect fluids from the flow through the wound dressing 10. In some embodiments, the absorbent material 5 may adhere to the wound dressing by an adhesive layer associated with the second material layer 3, or the absorbent material may remain in place over the wound by applying adhesive tape or another for of bandage over the absorbent material 5.

Some embodiments may include the step of removing the absorbent material 5 without disturbing the wound dressing 10. Thus, the absorbent material 5 may be changed without exposing the wound to the environment, because the wound dressing 10 may remain in place while used absorbent material is removed and fresh unused absorbent material is applied to the wound.

Still, further embodiments include the step administering therapeutics through the wound dressing. In some embodiments, a topical composition including one or more therapeutic agents, such as those described above, may be applied to the outer surface of the wound dressing while the position of the wound dressing over the wound is maintained. The therapeutic agents may be carried directly to the wound through the dressing without removing the dressing and exposing the wound to the environment. Embodiments are not limited to specific therapeutic agents, and the material used to prepare the first material layer 2 and the second material layer 3 may be selected to provide an effective flow of therapeutics through the wound dressing. Similarly, any type of topical composition including, but not limited to, gels, pastes, ointments, creams, emulsions, suspensions, or the like containing the therapeutic agent may be applied.

The wound dressing and methods can be used to treat any type of wound, including lacerations, cuts, scrapes. abrasion, post-operative wounds, denuded skin, burns, and the like. The wound dressing can remain in contact with the wound throughout the healing process because therapeutic agents can be administered topically through the wound dressing of the invention an absorbent “bandages” can be changed without removing the wound dressing of the invention. This provides a means for reducing exposure of the wound to the environment while allowing free flow of air, fluids, nutrients, and therapeutics into and out of the wound. The anti-microbial activity of the activated carbon layer also reduces the likelihood of infection by trapping and eradicating microbes in the wound at the time the wound dressing is applied and throughout the healing process. by immobilizing microbes before they can reach the wound itself.

Although the present invention has been disclosed above, the disclosure does not the present invention. Persons having ordinary skill in the art can make any changes or modifications without departing from the spirit and scope of the present invention. Consequently, the scope of protection of the present invention is based on the claims attached.

1. A wound dressing comprising:
a first material layer;
an activated carbon layer; and
a second material layer,
wherein at least one of the first material and the second material layer are impermeable.

2. The wound dressing of claim 1, wherein the activated carbon layer comprises an activated carbon cloth.

3. The wound dressing of claim 1, wherein the activated carbon layer comprises a woven cloth, non-woven cloth, knitted cloth, activated, carbon felt, or combinations thereof.

4. The wound dressing of claim 1, wherein the activated carbon layer comprises activated carbon particles, activated carbon powder, activated carbon fiber, or combinations thereof.

5. The wound dressing of claim 1, wherein the activated carbon layer comprises no noble metals.

6. The wound dressing of claim 1, wherein the activated carbon layer further comprises at least one therapeutic agent.

7. The wound dressing of claim 1, wherein the first material layer and the second material layer each independently comprise a polymeric material.

8. The wound dressing of claim 1, wherein the first material layer and the second material layer each independently comprise nylon, polyvinylidene fluoride (PVDF) or polytetrafluoroethylene (PTFE), ethylene methyl acrylate (EMA), polyvinyl chloride, polyvinyl dichloride, polyurea, polyolefin, ethylene vinylacetate copolymer, polyethylene, polypropylene, polyesters, polyethylene terephthlate, or combinations thereof.

9. The wound dressing of claim 1, wherein the first material layer and the second material layer independently comprise about 5 perforations/cm² to about 50 perforations/cm².

10. The wound dressing of claim 1, wherein the first material layer and the second material layer independently comprise perforations having an area of from about 0.01 mm² to about 2.0 mm².

11. The wound dressing of claim 1, wherein the first material layer and the second material layer independently comprise a hole-to-land ratio of from about 0.01 to about 1.0.

12. The wound dressing of claim 1, wherein the first material layer and the second material layer each independently comprise texturing.

13. The wound dressing of claim 1, further comprising an adhesive layer on an outer surface of at least the first material layer.

14. The wound dressing of claim 1, further comprising an adhesive layer on the first material layer and second material layer.

15. The wound dressing of claim 14, wherein an adhesive layer comprises acrylate ester copolymers, polyvinyl ethyl ether, polyurethane, or combinations thereof.

16. The wound dressing of claim 1, wherein the wound dressing does not include an absorbent material.

17. The wound dressing of claim 1, further comprising a cover layer.

18. A method for making a wound dressing comprising:
sealing an activated carbon layer between as first material layer and as second material layer.

19. The method of claim 18, further comprising applying an adhesive layer to at least one of the first material layer and the second material layer.

20. The method of claim 19, further comprising covering the adhesive layer with a cover layer,

21. The method of claim 18, wherein the activated carbon layer comprises a woven cloth, non-woven cloth, knitted cloth, activated carbon felt, or combinations thereof.

22. The method of claim 18, further comprising providing at least one therapeutic agent to the activated carbon layer before sealing.

23. The method of claim 18, wherein the activated carbon layer comprises no noble metals.

24. The method of claim 18, wherein the first material layer and the second material layer independently comprise nylon, polyvinylidene fluoride (PVDF), polytetrafluoroethylene (PTFE), ethylene methyl acrylate (EMA), polyvinyl chloride, polyvinyl dichloride, polyurea, polyolefin, ethylene vinylacetate copolymer, polyethylene, polypropylene, polyesters, polyethylene terephthlate, or combinations thereof.

25. The method of claim 18, wherein the first material layer and the second material layer independently comprise about 5 perforations/cm² to about 50 perforations/cm².

26. The method of claim 18, wherein the first material layer and the second material layer independently comprise perforations having an area of from about 0.01 mm² to about 2.0 mm².

27. The method of claim 18, wherein the first material layer and the second material layer independently comprise a hole-to-land ratio of from about 0.01 to about 1.0.

28. The method of claim 18, further comprising independently texturizing either of the first material layer, the second material layer, or a combination thereof.

29. A method for treating a wound comprising contacting a wound dressing comprising an activated carbon layer sealed between a first material layer and a second material layer to a wound.

30. The method of claim 29, further comprising applying an adsorbent material over the wound dressing.

31. The method of claim 30, further comprising:
removing the adsorbent material while the wound dressing remains affixed to the wound; and
applying a second absorbent material over the wound.

32. The method of claim 31, further comprising administering a therapeutic agent through the wound dressing before applying a second absorbent material.

33. The method of claim 32, wherein the therapeutic agent comprises a gel, paste, ointment, cream, emulsion, suspension, or combination thereof.

34. The method of claim 29, wherein the wound comprises a laceration, cut, scrape, abrasion, post-operative wound, denuded skin, burn, an allergic reaction, or combinations thereof.

35. The method of claim 29, further comprising affixing the wound dressing to the wound, tissue surrounding the wound, or a combination thereof.

36. The method of claim 35, wherein affixing comprises applying pressure to the wound dressing to effectuate bonding of an adhesive provided on the wound dressing.

37. The method of claim 35, wherein affixing comprises applying adhesive tape or another bandage over the wound dressing.

38. A wound dressing consisting of activated carbon cloth.

39. The wound dressing of claim 38, further comprising a material layer contacting the activated carbon cloth.

40. A method for treating a wound comprising contacting the wound with an activated carbon cloth.
